(19) United States
(12) Patent Application Publication
(10) Pub. No.: US 2003/0185910 A1
(43) Pub. Date: Oct. 2, 2003

(54) Cancer preventive composition comprising ginsenoside glycosides of red ginseng

(76) Inventors: Taik Koo Yun, Seoul (KR); Weon Seon Hong, Seoul (KR)

Correspondence Address: BIRCH STEWART KOLASCH & BIRCH, PO BOX 747, FALLS CHURCH, VA, 22040-0747, US

(21) Appl. No.: 10/067,760

(22) Filed: Feb. 8, 2002

(51) Int. Cl. A61K 35/78

(52) U.S. Cl. 424/728

(57) ABSTRACT

The present invention relates to a composition which contains as an active ingredient a ginsenoside glycoside (one or more component selected from the group consisting of ginsenoside Rg₃, ginsenoside Rg₅ and ginsenoside Rh₂) or a ginseng extract in combination with a ginsenoside glycoside (one or more component selected from the group consisting of ginsenoside Rg₃, ginsenoside Rg₅ and ginsenoside Rh₂), together with an adjuvant acceptable in the pharmaceutical or sitological field, and is formulated in the preparation form acceptable in the pharmaceutical or food purpose field by a method acceptable in the pharmaceutical or food chemistry. The composition of the present invention has a superior cancer-preventive effect on cancers occurring in individuals and populations having a high risk rate of cancer.

### DETAILED DESCRIPTION OF INVENTION

### PURPOSE OF THE INVENTION

### TECHNICAL FIELD TO WHICH THE INVENTION BELONGS AND THE PRIOR ART IN THIS FIELD

The present invention relates to a cancer preventive composition comprising a ginsenoside glycoside as an active ingredient.

In spite of the fact that about 50 kinds of cancer chemotherapeutic agents including alkylating agents first developed as the cancer chemotherapeutic agent at 1945 have been developed during past 50 years, cancer is still regarded as belonging to intractable diseases.

In the middle of the 1970's, the effort to increase the 5-years survival rate of cancer patients by one of two has also been ruined. Thus, the present inventor has started to study in 1977 and observe whether ginseng, which has been known as a miraculous medicine in the field of Chinese medicine, has a cancer preventive effect, on the assumption that cancer must be overcome by chemoprevention.

The extract of red ginseng was dissolved in drinking water and administered to NIH (GP) mice 3 weeks after a single subcutaneous injection of chemical carcinogens such as 9,10-dimethyl-1,2-benzanthracene (DMBA), urethane, aflatoxin B₁, N-2-fluorenylacetamide (FAA) and tobacco smoke condensates to newborn mice. At 28 weeks after administration of urethane combined with red ginseng, mice occurred a 22% (p<0.05) decrease of pulmonary adenoma while at 56 weeks after administration of aflatoxin combined with ginseng mice occurred a 75% decrease (p<0.05) of hepatoma. These results raised a hope that since a long-term administration of the extract of red ginseng inhibits tumor induced by urethane and aflatoxin, the intake of natural products may prevent cancer in human being. The results are described in the following Table 1 (Yun, T-K. et al.: Proc. 3rd Int. Ginseng Symp., 87-112, 1980, Seoul; Cancer Detect. Prev. 6:515-525, 1983;).

TABLE 1
Effects of the extract of red ginseng on mouse pulmonary adenoma
induced by carcinogens in long-term animal experiment

| | Sacri- | | Incidence of | | |
|---|---|---|---|---|---|
| | fice | Weight | pulmonary | Diffuse | Incidence of |
| Carcinogen | (wk) | of lung | adenoma | infiltration | hepatoma |
| DMBA* | 48 | 21% | — | 63% | — |
| | | decrease | | decrease | |
| Urethane | 28 | — | 22% | — | — |
| | | | decrease** | | |
| Aflatoxin B₁ | 56 | — | 29% | — | 75% |
| | | | decrease | | decrease** |

*DMBA = 9,10-dimethyl-1,2-benzanthracene
**p<0.05

1. Establishment of 9-Week Medium-term Anticarcinogenecity Test

The present inventor established the test method wherein mouse pulmonary adenoma is induced at 9 weeks using a carcinogen, benzo(a)pyrene, (termed Yun's model in the paper) and identified that anticarcinogenecity observed from the extract of 6-years root of red ginseng but not from 4-years root of fresh ginseng. Precisely, it was observed that 4-years root of ginseng not processed with heat has no cancer-preventive effect but 6-years root of red ginseng processed with heat exhibits cancer-preventive effect (Yun, T-K. et al.: J. Korean Cancer Ass. 19: 1-7, 1987; Anticancer Res., 15: 839-45, 1995).

2. Anticarcinogenecity Depending on Types and Ages of Ginseng

Thereafter, the present inventor experimented the cancer-preventive effect of ginseng depending on ages, i.e. 1.5, 3, 4, 5 and 6-years roots and types, i.e. powder and extracts of fresh ginseng, white ginseng and red ginseng, according to the above-defined 9-week medium-term anticarcinogenicity test. As a result, it has been observed that a significant anticarcinogenic effect is exhibited by the powder or extract of 6-years fresh ginseng, 4 and 5-years white ginseng, and 4, 5 and 6-years red ginseng. The result thereof is presented in the following Table 2. That is, when the age of ginseng is high, ginseng exhibits the cancer-preventive activity even though it is not processed with heat. Contrary to this, even if the age of ginseng is low, ginseng processed with heat exhibits the cancer-preventive activity starting from 4-years root, and at the same time, its powder and extract show the same cancer-preventive activity (Yun, T-K. et al.: Korean J. Ginseng Sci. 18:89-94, 1994 and 18:160-164, 1994; Acta Pharm. Sinica 17: 293-8, 1996).

TABLE 2
Anticarcinogenic effects of Panax ginseng C.A. Meyer depending on
types and ages based on 9-week medium-term anticarcinogenecity test

| | Incidence of lung adenoma | | | | | |
|---|---|---|---|---|---|---|
| Age of ginseng | Fresh ginseng | | White ginseng | | Red ginseng | |
| (years) | Powder | Extract | Powder | extract | powder | Extract |
| Benzo(a)pyrene | 41.3 | 63.9 | 45.0 | 41.3 | 48.6 | 47.5 |
| (BP) | | | | | | |
| BP + 1.5 | 31.2 | 48.3 | — | — | 37.9 | 40.7 |
| BP + 3 | 30.0 | 52.5 | 41.3 | 32.0 | 41.7 | 35.0 |
| BP + 4 | 31.3 | 51.8 | 38.0 | 46.0 | 31.7^{a} | 30.1^{a} |
| BP + 5 | 30.3 | 47.5 | 31.6^{a} | 44.0 | 28.3^{b} | 30.0 |
| BP + 6 | 27.8^{a} | 44.1^{a} | 25.3^{c} | 26.5^{a} | 25.4^{c} | 26.3^{a} |

BP Benzo(a)pyrene, ^{a}p<0.05, ^{b}p<0.02 and ^{c}p<0.01,

BP: Benzo(a)pyrene, ^{a}: p<0.05, ^{b}: p<0.02 and ^{c}: p<0.01,

3. Epidemiological Study in Human: (1) 905 Pairs of Cancer Case—Control Study

To investigate the effect of ginseng intake on the risk of cancer the case-control study consisting of 905 cancer patients and 905 non-cancer patients matched by age, sex and date of admission was conducted. As a result, the odds ratio of cancer was 0.56 (95% confidence interval: 0.45-0.69), and ginseng extract and white ginseng powder showed a more significant decrease in odds ratio in comparison to sliced fresh ginseng, ginseng juice or ginseng tea. Thus, as red ginseng produced by heat treatment of ginseng exhibits the most significant cancer-preventive effect in the animal experiment, it has been also identified in the epidemiological study in human that the intake of fresh ginseng juice or slice does not significantly decrease the odds ratio of cancer but the intake of heat-treated ginseng such as fresh ginseng extract with hot water or white ginseng extract with hot water provides a significant cancer-preventive effect. The result is presented in the following Table 3 (Yun, T-K. et al.: Int. J. Epidemiol. 19:871-876, 1990).

TABLE 3
Comparison of odds ratios in two cancer case-control studies with ginseng
intake

| | 905 pairs | | 1987 pairs | |
|---|---|---|---|---|
| | | 95% | | 95% |
| | Odds ratio | confidence | Odds ratio | confidence |
| Type of ginseng | (OR) | interval | (OR) | interval |
| Not intake of ginseng | 1.00 | Reference | 1.00 | Reference |
| Intake of ginseng | 0.56 | 0.46-0.69 | 0.50 | 0.44-0.58 |
| Fresh ginseng and | 0.74 | 0.53-1.04 | 0.79 | 0.63-1.01 |
| slice | | | | |
| Juice | 0.77 | 0.46-1.30 | 0.71 | 0.49-1.03 |
| Extract with hot water | 0.14 | 0.07-0.26 | 0.37 | 0.29-0.46 |
| White ginseng | 0.44 | 0.26-0.75 | 0.30 | 0.22-0.41 |
| powder | | | | |
| White ginseng extract | 0.64 | 0.50-0.82 | 0.57 | 0.48-0.68 |
| with hot water | | | | |
| Ginseng tea | 0.93 | 0.53-1.61 | 0.69 | 0.45-1.07 |
| Red ginseng extract | 0.45 | 0.05-3.32 | 0.20 | 0.08-0.50 |
| with hot water | | | | |
| Ginseng mixture | 0.27 | 0.13-0.53 | 0.16 | 0.10-0.25 |

Note: Odds ratio: Adjusted for age, sex, marital status, education, smoking and alcohol consumption

4. Epidemiological Study in Human: (2) 1987 Pairs of Cancer Case—Control Study

In order to explore 1) the types of ginseng products that have the most prominent cancer preventive effect, 2) the reproducibility of the dose-response relationship depending on the intake of ginseng, 3) the duration of ginseng consumption that has a significant cancer preventive effect, and 4) the types of cancer which can be prevented by ginseng, the present inventor extended the number of subjects for a case-control study on 1987 pairs. As in the result of the study in 905 pairs, the present study also presented the odds ratio (OR) depending on the types of ginseng to be 0.37 for intakers of fresh ginseng extract with hot water, 0.30 for intakers of white ginseng powder, 0.57 for intakers of white ginseng extract with hot water and 0.20 for intakers of red ginseng extract with hot water. Therefore, intakers of red ginseng products which are most strongly subjected to repeated heat treatment showed a great decrease in odds ratio and intakers of fresh ginseng slice, fresh ginseng juice, etc., as the raw ginseng, however, showed no significant decrease in odds ratio. The result is presented in Table 3.

Further, on the site of cancer, the odds ratios were significantly decreased for cancer of oral cavity, esophagus cancer, stomach cancer, colorectal cancer, liver cancer, pancreatic cancer, laryngeal cancer, lung cancer and other cancers. The result is presented in the following Table 4 (Yun, T-K. et al.: Cancer Epidemiol. Biomarkers & Prev. 4:401-408, 1995).

TABLE 4
Odds ratios (ORs) for various cancers in the cancer case-control study on
1987 pairs

| | Cancer cases | Controls | Odds | |
|---|---|---|---|---|
| | Never taken/ | Never taken/ | ratio | 95% confidence |
| Site of cancers | ever taken | ever taken | (OR) | interval (CI) |
| Lip, oral cavity | 67/92 | 40/119 | 0.47 | 0.29 ± 0.76 |
| and pharynx | | | | |
| Esophagus | 40/47 | 14/73 | 0.20 | 0.09 ± 0.38 |
| Stomach | 142/158 | 76/224 | 0.36 | 0.09 ± 0.52 |
| Colon and rectum | 55/63 | 32/86 | 0.42 | 0.24 ± 0.74 |
| Liver | 108/156 | 67/197 | 0.48 | 0.33 ± 0.70 |
| Pancreas | 12/11 | 5/18 | 0.22 | 0.05 ± 0.95 |
| Larynx | 21/19 | 8/32 | 0.18 | 0.06 ± 0.54 |
| Lung | 120/156 | 81/195 | 0.55 | 0.38 ± 0.79 |
| Breast | 82/92 | 70/109 | 0.63 | 0.40 ± 1.05 |
| Cervix uteri | 156/146 | 312/170 | 0.72 | 0.52 ± 1.01 |
| Ovary | 17/5 | 8/14 | 0.15 | 0.04 ± 0.60 |
| Urinary bladder | 23/40 | 16/47 | 0.64 | 0.28 ± 1.47 |
| Thyroid gland | 16/24 | 14/26 | 0.96 | 0.38 ± 2.44 |
| Others | 53/61 | 35/79 | 0.48 | 0.27 ± 0.85 |

Note: Odds ratio: Adjusted for age, sex, marital status, education, smoking and alcohol consumption

5. Epidemiological Study in Human: Prospective Study on Populations Residing on a Ginseng Cultivation Area

The present inventor conducted a more reliable cohort study. Specifically, the study was carried out for 5 years on a total of 4,634 subjects consisting of 2,362 males and 2,272 females who completed a questionnaire on ginseng intake, over 40 years old residing in Kangwha-eup, as one of main ginseng cultivation areas in Korea. As a result, among the subjects, 355 cases (7.7%) had died and the death caused by cancer was observed for 79 cases (22.8%). The total number of cases occurring cancer accounted for 137 (3.0%) with 58 (1.3%) alive and 79 (1.7%) deaths. A total of participants eligible for the analysis accounted for 4,634 subjects of which 3,267 subjects (70.5%) were ginseng consumers. The ginseng consumers had the odds ratio (OR) of 0.40 (95% CI 0.28-0.56) compared with non-consumers. On the type of ginseng, the odds ratio was 0.31 (95% CI: 0.13-0.74) for consumers of fresh ginseng extract with hot water and 0.34 (95% CI: 0.20-0.53) for consumers of the combination of various ginsengs. There was no case occurring cancer among 24 consumers of red ginseng extract with hot water.

There was a dose-response relationship wherein the odds rate of cancer is decreased with increasing the frequency of ginseng intake. 137 cancer cases included 42 cases of stomach cancer, 24 cases of lung cancer, 14 cases of liver cancer and 57 cases of other cancers. The result thereof is presented in the following Table 5. The odds ratios of ginseng consumers were 0.33 (95% CI: 0.18-0.57) for stomach cancer and 0.30 (95% CI: 0.14-0.65) for lung cancer. On the types of ginseng, the consumers of fresh ginseng extract with hot water showed the odds rate of 0.33 (95% CI: 0.12-0.88). It was considered that these results strongly suggest that Panax ginseng C. A. Meyer (Korean ginseng) has a non-toxic, non-organ specific preventive effect against cancer. In the present study, since the odds ratios of all cancers were also decreased for consumers of fresh ginseng extract with hot water and further, among 24 consumers of red ginseng products there was no death from cancer, it had been assumed that if ginseng is subjected to heat treatment and then consumed, unique ingredients are produced and may exhibit the preventive effect against cancer (Yun, T-K. et al.: Int. J. Epidemiol. 27:359-364, 1998).

TABLE 5
Cohort study on the relationship between ginseng intake and the risk of cancers in
populations residing in Kangwha-eup

| | | Cancers frequently occurring in populations residing in Kangwha-eup | | | | | | | | |
|---|---|---|---|---|---|---|---|---|---|---|
| | No. of | Stomach cancer (42) | | | Lung cancer (24) | | | Liver cancer (14) | | |
| Type of ginseng | subjects | No. | OR | 95% CI | No. | OR | 95% CI | No. | OR | 95% CI |
| No intake | 1283 | 23 | 1.00 | — | 14 | 1.00 | — | 4 | 1 | — |
| Ginseng intake | 3167 | 19 | 0.33 | 0.18-0.57 | 10 | 0.30 | 0.14-0.65 | 10 | 0.86 | 0.25-2.94 |
| Fresh ginseng | | | | | | | | | | |
| Slice or juice | 236 | 2 | 0.57 | 0.17-1.94 | 1 | 0.67 | 0.15-3.43 | 2 | 1.97 | 0.34-2.95 |
| Extract with hot | 296 | 1 | 0.33 | 0.12-0.88 | 1 | 0.28 | 0.04-2.17 | — | — | — |
| Water | | | | | | | | | | |
| White ginseng | | | | | | | | | | |
| Powder | 147 | 1 | 0.24 | 0.03-1.84 | — | — | — | — | — | — |
| Extract with hot | 68 | 2 | 1.34 | 0.30-5.97 | — | — | — | — | — | — |
| Water | | | | | | | | | | |
| Tea | 442 | 6 | 0.64 | 0.26-1.61 | 4 | 0.80 | 0.26-2.44 | 2 | 1.72 | 0.15-4.87 |
| Boiled chicken with | 381 | 5 | 0.43 | 0.12-1.43 | 1 | 0.35 | 0.08-1.95 | 1 | 0.85 | 0.15-4.87 |
| young ginseng root | | | | | | | | | | |

Note:
Odds rate (OR): Adjusted for age, sex, education, smoking and alcohol consumption.
95% CI: 95% confidence interval
Values in parentheses indicate number of cancer cases.

### Technical Subject to be Accomplished by the Invention

The present inventor has extensively studied the cancer preventive effect of red ginseng prepared by heat treatment of fresh ginseng and drying, on the basis of the results of the study up to the present which demonstrate that among fresh ginseng, white ginseng and red ginseng and the products produced therefrom as various processed forms of ginseng the heat-treated products have a significant potent cancer preventive effect in various animal experiments and epidemiological studies on human.

As ginseng glycosides ginsenoside Rg₃, ginsenoside Rg₅ and ginsenoside Rh₂ are the known materials of which the extraction method and structural determination have been described in the following references: Eyakov, G. B. et al.: Synthesis of the ginseng glycosides and their analogs. Proceedings of 6th International Ginseng Symposium. P74-83, 1993; and Anufriev, V. P. et al.: Synthesis of ginsenoside Rg₃, a minor constituent of Ginseng radix. Carbohydrate Research 304: p179-182, 1997. The chemical structures of those ginsenosides are represented by the following formulas:

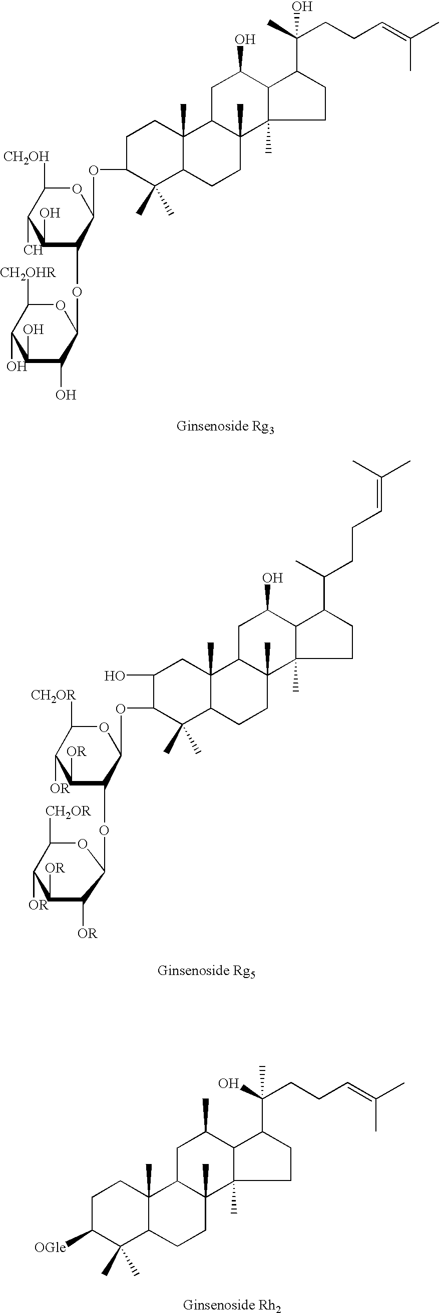

Ginsenoside Rg₃ Ginsenoside Rg₅ Ginsenoside Rh₂

Those ginsenoside glycosides are contained in ginseng, particularly in red ginseng, in a minor amount, and thus, have been synthetically or semi-synthetically prepared at present.

However, it has never been disclosed as yet that these ginseng glycosides have a cancer preventive effect.

The present inventor has discovered the experimental findings that those ginsenoside glycosides have a superior preventive effect on cancer.

Therefore, the purpose of the present invention is to provide a pharmaceutical composition for prevention of cancer, which comprises as an active ingredient one or more glycosides selected from the group consisting of ginsenoside glycosides (ginsenoside Rg₃, ginsenoside Rg₅ and ginsenoside Rh₂).

Another purpose of the present invention is to provide a food composition for prevention of cancer, which comprises as an active ingredient one or more glycosides selected from the group consisting of ginsenoside glycosides (ginsenoside Rg₃, ginsenoside Rg₅ and ginsenoside Rh₂).

These ginsenoside glycosides are contained in red ginseng only in a minor amount (less than about 0.01 wt %). Therefore, if such glycosides are incorporated into the extract of red ginseng, the cancer preventive effect of red ginseng can be multiplied.

The present inventor has discovered that the addition of one or more glycosides selected from the group consisting of ginsenoside glycosides (ginsenoside Rg₃, ginsenoside Rg₅ and ginsenoside Rh₂) to the extract of red ginseng can further augment the cancer preventive effect of the red ginseng extract.

Therefore, further purpose of the present invention is to provide a pharmaceutical composition for prevention of cancer which comprises as an active ingredient a red ginseng extract combined with one or more glycosides selected from the group consisting of ginsenoside glycosides (ginsenoside Rg₃, ginsenoside Rg₅ and ginsenoside Rh₂) so that the cancer preventive effect of red ginseng extract can be further augmented.

Still another purpose of the present invention is to provide a food composition for prevention of cancer which comprises as an active ingredient a red ginseng extract combined with one or more glycosides selected from the group consisting of ginsenoside glycosides (ginsenoside Rg₃, ginsenoside Rg₅ and ginsenoside Rh₂) so that the cancer preventive effect of red ginseng extract can be further augmented.

### Constitution of Invention

In the present invention it has been identified that ginsenoside Rg₃, ginsenoside Rg₅ and ginsenoside Rh₂ as a unique saponin present in red ginseng prepared by heat treatment of fresh ginseng and drying have the cancer preventive effect. Thus, the present invention provides the pharmaceutical composition for prevention of cancer which comprises such ginsenoside compound, and further the food composition for prevention of cancer which comprises ginsenoside Rg₃, ginsenoside Rg₅ and ginsenoside Rh₂.

Ginsenoside Rg₃, ginsenoside Rg₅ and ginsenoside Rh₂ can be extracted directly from red ginseng or by heat treatment of fresh ginseng or white ginseng, or can be synthesized by the known synthetic method (Eyakov, G. B. et al.: Synthesis of the ginseng glycosides and their analogs. Proceedings of 6th International Ginseng Symposium. P74-83, 1993; and Anufriev, V. P. et al.: Synthesis of ginsenoside Rg₃, a minor constituent of Ginseng radix. Carbohydrate Research 304: p179-182, 1997).

In order to prevent the occurrence of cancer, ginsenoside Rg₃, ginsenoside Rg₅ and ginsenoside Rh₂ as the active ingredient are mixed with the carrier acceptable to pharmaceutical purpose or as the food additive to prepare the cancer preventive composition. This composition is formulated in the form of a preparation conventionally acceptable to pharmaceutical or food purpose using diluents, disintegrating agents, excipients, gliding agents, sweeteners, flavoring agents, binders, solubilizing agents, dispersing agents, stabilizing agents, suspending agents, lubricants, preserving agents, pigments, painless agents, etc. conventionally acceptable to pharmaceutical or food purpose by the method conventionally acceptable in the pharmaceutical and food industry.

Such forms of the preparation include injections, tablets, powders, granules, capsules, suspensions, syrups, solutions, or parenterally administrable preparations in the form of a unit dosage form or a multiple dosage form. In addition, for the purpose of development of health foods for cancer prevention the composition of the present invention can be incorporated into the food product, for example, various foods, beverages, vitamin complexes, etc.

Although the cancer preventive composition containing ginsenoside Rg₃, ginsenoside Rg₅ and ginsenoside Rh₂ as the active ingredient can be administered to adult generally in a daily amount of 1.0 mg to 500 mg, it is administered preferably in an amount of 10 mg to 100 mg once or with dividing over several times.

It has been discovered that up to the present ginsenoside Rg₃, ginsenoside Rg₅ and ginsenoside Rh₂ according to the present invention did not exhibit any acute toxicity when they are administered to mice via oral route in an amount of 1000 mg/kg in the toxicity test, and therefore, have substantially no toxicity.

The present invention will be more specifically illustrated through the following examples. It should be understood that these examples are intended only to specifically explain the present invention and the scope of the present invention is not limited by these examples in any manner.

### EXPERIMENT 1

### Test for Cancer Preventive Effect of Ginsenoside Rg₃, Ginsenoside Rg₅ and Ginsenoside Rh₂ Based on a 9-Weeks Medium-term Anticarcinogenicity Test

1. Test method:

1) Experimental animal: In this experiment newborn non-inbred strain NIH(GP) mice were used. Each experimental animal was in a breeding chamber maintained at constant temperature and humidity and, after weaning, given a solid feed prepared according to the NIH-7 open formula. The experimental animals were allowed to freely take drinking water and diet.

2) Establishment of the test groups: The test groups were a total of 10 groups including the normal control group, the ginsenoside Rg₃ treatment group, the ginsenoside Rg₅ group, the ginsenoside Rh₁ group, the ginsenoside Rh₂ group, the BP 0.5 mg group, the BP plus ginsenoside Rg₃ group, the BP plus ginsenoside Rg₅ group, the BP plus ginsenoside Rh₁ group, and the BP plus ginsenoside Rh₂ group, wherein 25 to 30 newborn mouse, female and male respectively, were used in each group.

3) Chemical carcinogen: As the chemical carcinogen, benzo(a)pyrene (BP: Sigma Chemical Co.) was used. The given amount of BP was suspended in 1% aqueous gelatin solution and then injected subcutaneously in the subscapular region once in an amount of 0.02 cc (0.5 mg).

4) Administration of respective ginsenosides: Respective ginsenosides, i.e. ginsenoside Rg₃, ginsenoside Rg₅, ginsenoside Rh₁ and ginsenoside Rh₂ were suspended in drinking water to 80 μg/ml and daily administered from the 3rd week to 9 th week after birth, i.e. during the period of 6 weeks.

5) Monitoring and sacrifice of experimental animals: The appearance of mice was monitored every time at which the feed and drinking water were changed, beginning from weaning at the 3th week after birth. Mice were weighed at intervals of one week and sacrificed at the 9th week after birth and then subjected to autopsy.

6) Pathological investigation: Immediately after autopsy, lungs were exposed to observe the presence or absence of nodules of lung adenoma, and at the same time, to observe the presence or absence of any lesion in respective main organs by the naked eye. Then lungs were fixed in Tellyesniczky solution and the other main organs were fixed in 10% neutral formalin solution, cut and then examined with a microscope. After fixing of lungs, nodules of long adenoma were counted by the naked eye. Then lungs were cut as in case of other main organs, stained with hematoxylin-eosin and then investigated to find nodules of lung adenoma

7) Statistical treatment: The comparisons of the calculated lung adenoma incidence in respective test groups were made using the Chi-square test and the comparisons of multiplicity were made using Student's t test to obtain P-value.

2. Determination on Cancer Preventive Effect of Saponins Unique to Red Ginseng

As shown in the following Table 6, a total of 10 groups including the normal control group, the ginsenoside Rg₃ treatment group, the ginsenoside Rg5 group, the ginsenoside Rh₁ group, the ginsenoside Rh₂ group, the BP 0.5 mg group, the BP plus ginsenoside Rg₃ group, the BP plus ginsenoside Rg₅ group, the BP plus ginsenoside Rh₁ group, and the BP plus ginsenoside Rh₂ group were established as the test groups and in each group 25 to 30 newborn mice, female and male respectively, were used to conduct the experiment. As the result, since a total of 5 groups including the normal control group, the ginsenoside Rg₃ treatment group, the ginsenoside Rg₅ group, the ginsenoside Rh₁ group, the ginsenoside Rh₂ group and the red ginseng extract group did never occur lung adenoma, it can be seen that the experiment using singularly each of the red ginseng extract and three kinds of ginsenosides, i.e. ginsenoside Rg₃, ginsenoside Rg₅ and ginsenoside Rh₂ group does not cause any carcinogenicity. However, in mice which were subcutaneously given with 0.5 mg of benzo(a)pyrene (BP) within 24 hours after birth and then sacrificed at the 9th week after birth the incidence of lung adenoma reached 60% on the average. On the other hand, the BP plus ginsenoside Rg₃ group showed a statistically significant decrease in the incidence of lung adenoma to 46.7% on the average and the BP plus ginsenoside Rg₅ group also showed a significant decrease in lung adenoma to 45.0%. Meanwhile, the BP plus ginsenoside Rh₂ group showed the lung adenoma incidence of 48.3% and therefore, had a tendency to decrease the lung adenoma incidence. This result had no statistical significance but showed a tendency of anticarcinogenicity.

The results from such 9-weeks anticarcinogenicity test recognized that among ginseng saponin compounds which can be prepared from fresh ginseng or red ginseng by extraction, separation, purification or chemical method, the composition containing at least of one of three kinds of compounds, i.e. ginsenoside Rg₃, ginsenoside Rg₅ and ginsenoside Rh₂ is suitable as the cancer preventive agent, particularly a chemical agent for prevention of cancer which can be developed naturally or in high risk populations. Further, anticarcinogenic effect of red ginseng was observed from a long-term experiment using mouse lung adenoma and the anticarcinogenicity was also demonstrated depending on the ages and types of ginseng in 9-weeks medium-term test. Subsequently, in the cancer case-control study on 905 pairs and another cancer case-control study on 1987 pairs and further, in the cohort study on 4636 people over 40 years residing in ginseng cultivation area, it has been commonly observed that in the consumers of the extract of heat-treated ginseng and the red ginseng products a high odds ratio is decreased and further, the odds ratios of various cancers are statistically significantly decreased in proportion to the frequency and amount of administration.

TABLE 6
Anticarcinogenic effect of three kinds of ginsenosides, i.e. ginsenoside Rg₃,
ginsenoside Rg₅ and ginsenoside Rh₂

| Test groups and | | Mouse | | | Multiplicity |
|---|---|---|---|---|---|
| treatment | Dose | Sex | No. | Incidence | (Mean ± S.D.) |
| Normal control | — | M | 25 | 0 | — |
| | | F | 25 | 0 | — |
| | | M + F | 50 | 0 | — |
| Red ginseng extract | 2 mg/ml | M | 25 | 0 | — |
| | | F | 25 | 0 | — |
| | | M + F | 50 | 0 | — |
| Ginsenoside Rg₃ | 80 μg/ml | M | 25 | 0 | — |
| | | F | 25 | 0 | — |
| | | M + F | 50 | 0 | — |
| Ginsenoside Rg₅ | 80 μg/ml | M | 25 | 0 | — |
| | | F | 25 | 0 | — |
| | | M + F | 50 | 0 | — |
| Ginsenoside Rh₂ | 80 μg/ml | M | 25 | 0 | — |
| | | F | 25 | 0 | — |
| | | M + F | 50 | 0 | — |
| Benzo(a)pyrene (BP) | 0.5 mg/head | M | 25 | 14 (56.0) | 1.20 ± 1.44 |
| | | F | 25 | 16 (64.0) | 1.80 ± 2.12 |
| | | M + F | 50 | 30 (60.0) | 1.50 ± 1.82 |
| BP + Ginsenoside Rg₃ | 0.5 mg/head | M | 30 | 13 (43.3) | 0.67 ± 0.96 |
| | 80 μg/ml | F | 30 | 15 (50.0) | 1.03 ± 1.27 |
| | | M + F | 60 | 28 (46.7) | 0.85 ± 1.13 |
| BP + Ginsenoside Rg₅ | 0.5 mg/head | M | 30 | 13 (43.3) | 0.83 ± 1.21 |
| | 80 μg/ml | F | 30 | 14 (46.7) | 1.33 ± 2.89 |
| | | M + F | 60 | 27 (45.0)* | 1.08 ± 2.21 |
| BP + Ginsenoside Rh₁ | 0.5 mg/head | M | 30 | 15 (50.0) | 1.20 ± 1.54 |
| | 80 μg/ml | F | 30 | 16 (53.3) | 1.49 ± 1.86 |
| | | M + F | 60 | 31 (51.7) | 1.03 ± 1.27 |
| BP + Ginsenoside Rh₂ | 0.5 mg/head | M | 30 | 13 (43.3) | 0.77 ± 1.14 |
| | 80 μg/ml | F | 30 | 16 (53.3) | 1.53 ± 1.93 |
| | | M + F | 60 | 29 (48.3) | 1.15 ± 1.61 |

*P <0.05

### EXPERIMENT 2

### Acute Toxicity Test of Ginsenoside Rg₃, Ginsenoside Rg₅ and Ginsenoside Rh₂

1. Test Method

20 male ICR mice weighing 26-30 grams were used in each test group to conduct the acute toxicity test of ginsenosides Rg₃, Rg₅ and Rh₂. Specifically, each of ginsenosides Rg₃, Rg₅ and Rh₂ was prepared in 0.5% Tween 80 as the solvent to the concentration of 100 mg/ml and then orally administered to mice once in an amount of 0.2 ml per 20 grams of body weight. The control group was given only 0.5% Tween 80 solvent via oral route. The experimental animals were observed for 14 days after administration for whether any of animals is dead. Further, on 14th day after administration the animals were sacrificed and then autopsied to examine macroscopically the main organs.

2. Test Result

The purpose of the present test is to obtain the basic data for the toxicity of ginsenosides Rg₃, Rg₅ and Rh₂. As the result of the test, no dead animal was observed for 14 days at the dose level of 1000 mg/kg of of ginsenosides Rg₃, Rg₅ and Rh₂. Further, as the result of autopsy examination on 14th day after administration no special pathological finding were observed macroscopically. In conclusion, it has been determined that ginsenosides Rg₃, Rg₅ and Rh₂ have no toxicity when they are administered via oral route at the above dose level.

Therefore, ginsenoside glycosides of the present invention can be used for the purpose of cancer prevention.

### EXAMPLE 1

100 mg of red ginseng extract was well mixed with 1 mg of the mixture of ginsenosides Rg₃, Rg₅ and Rh₂ to obtain the mixture wherein ginsenoside glycodise are greatly enriched.

The present invention will be more specifically illustrated by the following preparation examples.

### PREPARATION EXAMPLE 1

| | | |
|---|---|---|
| Mixture of ginsenosides Rg₃, Rg₅ and Rh₂ | 5 | mg |
| Sterilized distilled water for injection | | q.s. |
| pH controlling agent | | q.s. |

The ginsenoside mixture was dissolved in distilled water for injection and then adjusted to about pH 7.6 with a pH controlling agent. Then, the whole mixture was made to the volume of 2 ml and then filled in a 2 ml ampoule, which was sterilized to prepare the desired injection preparation.

### PREPARATION EXAMPLE 2

| | | |
|---|---|---|
| Mixture of Example 1 | 50 | mg |
| Sterilized distilled water for injection | | q.s. |
| pH controlling agent | | q.s. |

The mixture of Example 1 was dissolved in distilled water for injection and then adjusted to about pH 7.2 with a pH controlling agent. Then, the whole mixture was made to the volume of 2 ml and then filled in a 2 ml ampoule, which was sterilized to prepare the injection preparation.

### PREPARATION EXAMPLE 3

| | | |
|---|---|---|
| Mixture of of ginsenosides Rg₃, Rg₅ and Rh₂ | 20 | mg |
| Lactose | 100 | mg |
| Magnesium stearate | | q.s. |

The above ingredients were mixed and then compressed according to the conventional method for preparing tablets to prepare the desired tablet preparation.

### PREPARATION EXAMPLE 4

| | | |
|---|---|---|
| Mixture of Example 1 | 50 | mg |
| Lactose | 100 | mg |
| Starch | 50 | mg |
| Magnesium stearate | | q.s. |

The above ingredients were mixed and then compressed according to the conventional method for preparing tablets to prepare the desired tablet preparation.

### PREPARATION EXAMPLE 5

| | | |
|---|---|---|
| Ginsenoside Rg₃ | 20 | mg |
| Lactose | 100 | mg |
| Starch | 50 | mg |
| Talc | 2 | mg |
| Magnesium stearate | | q.s. |

The above ingredients were mixed and then filled in a gelatin capsule according to the conventional method for preparing capsules to prepare the desired capsule preparation.

### PREPARATION EXAMPLE 6

| | | |
|---|---|---|
| Ginsenoside Rg₅ | 30 | mg |
| Lactose | 50 | mg |
| Starch | 50 | mg |
| Talc | 2 | mg |
| Magnesium stearate | | q.s. |

The above ingredients were mixed and then filled in a gelatin capsule according to the conventional method for preparing capsules to prepare the desired capsule preparation.

### PREPARATION EXAMPLE 7

| | | |
|---|---|---|
| Mixture of Example 1 | 100 | mg |
| Sucrose | 20 | g |
| Isomerized sugar | 20 | g |
| Lemon flavor | | q.s. |
| Purified water | to make 100 | ml. |

The above ingredients were mixed according to the conventional method for preparing solutions and then filled in a 100 ml brown bottle which was then sterilized to prepare the desired solution preparation.

### PREPARATION EXAMPLE 8

| | |
|---|---|
| Mixture of Example 1 | 100 mg |
| Lactose | 100 mg |

The above ingredients were mixed to prepare the desired powder preparation.

### PREPARATION EXAMPLE 9

| | | |
|---|---|---|
| Mixture of Example 1 | 100 | mg |
| Vitamin E | 1 | mg |
| Sucrose | 20 | g |
| Isomerized sugar | 20 | g |

| | |
|---|---|
| Lemon flavor | q.s. |
| Purified water | to make 100 ml. |

The above ingredients were mixed according to the conventional method for preparing solutions and then filled in a 100 ml brown bottle which was then sterilized to prepare the desired solution preparation.

### PREPARATION EXAMPLE 10

| | | |
|---|---|---|
| Mixture of ginsenosides Rg₃, Rg₅ and Rh₂ | 200 | mg |
| Vitamin E | 1 | mg |
| Vitamin C | 100 | mg |
| Sucrose | 20 | g |
| Isomerized sugar | 20 | g |
| Lemon flavor | | q.s. |
| Purified water | to make 100 | ml. |

What is claimed

1. A composition having cancer preventive effect, which comprises as an active ingredient a ginsenoside glycoside (one or more component selected from the group consisting of ginsenoside Rg₃, ginsenoside Rg₅ and ginsenoside Rh₂) together with an adjuvant acceptable to pharmaceutical or food purpose, and is formulated in the preparation form acceptable in the pharmaceutical or food purpose by a method acceptable in the field of pharmaceutical or food chemistry.

2. A composition having cancer preventive effect, which comprises as an active ingredient a ginseng extract in combination with a ginsenoside glycoside (one or more component selected from the group consisting of ginsenoside Rg₃, ginsenoside Rg₅ and ginsenoside Rh₂) together with an adjuvant acceptable to pharmaceutical or food purpose, and is formulated in the preparation form acceptable in the pharmaceutical or food purpose by a method acceptable in the field of pharmaceutical or food chemistry.

3. The composition having cancer preventive effect according to claim 2, which contains as an active ingredient the ginseng extract in combination with 0.05 to 20 wt % of the ginsenoside glycoside (one or more component selected from the group consisting of ginsenoside Rg₃, ginsenoside Rg₅ and ginsenoside Rh₂) together with an adjuvant acceptable to pharmaceutical or food purpose, and is formulated in the preparation form acceptable in the pharmaceutical or food purpose by a method acceptable in the field of pharmaceutical or food chemistry.

4. The composition having cancer preventive effect according to anyone of claims 1 to 3, which further contains a component selected from vitamins and amino acids.
